(19) United States
(12) Patent Application Publication
(10) Pub. No.: US 2025/0340917 A1
(43) Pub. Date: Nov. 6, 2025

(54) BIOSENSOR AND DETECTION METHOD FOR TARGET SUBSTANCE

(71) Applicant: POSTECH RESEARCH AND BUSINESS DEVELOPMENT FOUNDATION, Pohang-si (KR)

(72) Inventors: Seung Soo OH, Pohang-si (KR); Junwoo SON, Pohang-si (KR); Jin Min KIM, Pohang-si (KR); Min Guk JO, Pohang-si (KR)

(73) Assignee: POSTECH RESEARCH AND BUSINESS DEVELOPMENT FOUNDATION, Pohang-si (KR)

(21) Appl. No.: 19/092,250

(22) Filed: Mar. 27, 2025

(30) Foreign Application Priority Data
May 3, 2024 (KR) 10-2024-0059370

(51) Int. Cl. C12Q 1/00

(52) U.S. Cl. CPC C12Q 1/005

(57) ABSTRACT

The invention relates to a biosensor and a method of detecting a target substance by using the biosensor. The biosensor according to an embodiment of the invention includes a reaction layer generating a hydrogen ion through a physical and/or chemical interaction with a target substance in a specimen, a hydrogen ion transfer layer formed on one side of the reaction layer to transfer the hydrogen ion generated in the reaction layer, and a phase transition layer formed on one side of the hydrogen ion transfer layer, in which a phase of a substance changes due to the hydrogen ion transferred from the hydrogen ion transfer layer.

### CROSS-REFERENCE TO RELATED APPLICATION

This application claims the benefit of and priority to Korean Patent Application No. 10-2024-0059370, filed on May 3, 2024, the entire contents of which are incorporated herein by reference.

### BACKGROUND OF THE INVENTION

### 1. Field of the Invention

The present invention relates to a biosensor and a method of detecting a target substance by using the biosensor.

### 2. Description of the Related Art

For effective field diagnosis and personalized treatment, disease indicators should be consistently identified in a straightforward manner. At present, to sense abnormal physical conditions, electrical monitoring of vital signs such as a body temperature, a blood pressure, and a respiratory rate is routinely carried out on both patients and healthy individuals.

However, changes in vital signs may not be direct indicators of diseases but rather general symptoms of stress or exercise, and thus monitoring of various biomarkers such as ions, metabolic products, and proteins should be ensured in order to make it possible to carry out practical self-diagnosis.

The occurrence or recurrence of disease may change the dynamics of human prognostic biomarkers, and pathogens and viral infections may trigger innate immune responses, which leads to immediate changes in the levels of associated biomarkers. For example, glucose is regarded as the most important biomarker of metabolic products for diabetes because its millimolar concentration in the blood changes rapidly, indicating the risk of diabetes. Therefore, this facilitates diabetes management for subsequent treatment or urgent treatment. With the availability of various commercial glucose meters, better self-management and better protection functions are being provided to patients with diabetes and prediabetes based on rapid diagnosis of hypoglycemia or hyperglycemia.

In addition, body fluids contain a large number of prognostic biomarkers having significantly different concentration ranges. Furthermore, non-invasive clinical fluids such as saliva, tears, sweat, and urine serve as suitable biomarkers for field testing. However, the levels of biomarkers contained in samples collected non-invasively are about 10 to 10,000 times lower than the levels of biomarkers in the blood that are collected invasively. For example, the concentration of glutamate, which is a neurotransmitter biomarker for diagnosing neurodegenerative diseases, is markedly lower in saliva than in blood (950 nmol 1⁻¹ for healthy individuals, and 1,350 nmol 1⁻¹ for Alzheimer's patients). Such a low concentration of glutamate greatly exceeds the limits of detection of existing glutamate biosensors, which causes a problem in that it is difficult to apply existing glutamate biosensors to non-invasive samples. Moreover, in various clinical situations such as pediatric biopsies and multi-analyte analyses, the amount of samples is often minimized, and in such cases, there is an increasing need to detect biomarkers using small amounts of non-invasive samples.

As a result, various detection technologies have been studied to selectively recognize clinically significant biomarkers even at low concentrations.

Among these, significant attention has been paid to a miniaturized field effect transistor (FET) as a biomarker detector because even a slight difference in surface potential can cause a significant change in current. However, body fluids are complex mixtures that contain highly charged molecules such as nucleic acids, proteins, and metabolic products, and nonspecific surface adsorption by these substances often causes unwanted artifact signals, regardless of the presence of target molecules. In addition, a phenomenon (Debye shielding) in which the surface potential is easily neutralized may occur in a high-salt solution, which makes a FET biosensor insensitive to a biomarker without an unexpected electrical change.

Non-specific physical adsorption and surface potential screening cause a drastic decrease in reliability not only for a bio-FET but also for other types of diagnostic biosensors, and thus it is very difficult to amplify a specific signal that makes it possible to detect a target biomarker at an extremely low concentration without the purification and post-treatment of the specimen.

A biosensor is disclosed in Korean Patent Laid-open Gazette No. 2020-0019040.

### SUMMARY OF THE INVENTION

An object of the invention is to provide a biosensor that is capable of detecting a target substance at an extremely low concentration, and a detection method for a target substance.

However, the object to be achieved by the invention is not limited to those mentioned above, and the other objects not mentioned will be clearly understood by those skilled in the art from the description below.

One aspect according to the invention for achieving the above-described object is to provide a biosensor including:
a reaction layer that generates a hydrogen ion through a physical and/or chemical interaction with a target substance in a specimen;
a hydrogen ion transfer layer that is formed on one side of the reaction layer to transfer the hydrogen ion generated in the reaction layer; and
a phase transition layer that is formed on one side of the hydrogen ion transfer layer, in which a phase of a substance changes due to the hydrogen ion transferred from the hydrogen ion transfer layer.

In the biosensor according to one exemplary embodiment of the invention, a first electrode that is formed on one side of the phase transition layer, and a second electrode that is formed on the other side of the phase transition layer may be further included.

In the biosensor according to one exemplary embodiment of the invention, the reaction layer may contain a redox enzyme.

In the biosensor according to one exemplary embodiment of the invention, the reaction layer may contain one or more selected among a dehydrogenase, a peroxidase, a reductase, an oxidase, an oxygenase, and a hydroxylase.

In the biosensor according to one exemplary embodiment of the invention, the hydrogen ion transfer layer may contain a substance having proton conductivity that enables ion transfer while maintaining electronic insulation.

In the biosensor according to one exemplary embodiment of the invention, the hydrogen ion transfer layer may contain one or more selected among a proton conductive solid oxide, a proton conductive polymer substance, a perovskite proton conductor, a hydrogen-bonded organic framework (HOF), a substance functionalized with an acidic group or a protonic molecule, and a proton conductive ceramic.

In the biosensor according to one exemplary embodiment of the invention, the hydrogen ion transfer layer may have liquid impermeability.

In the biosensor according to one exemplary embodiment of the invention, the silicon oxide may be an amorphous SiO₂.

In the biosensor according to one exemplary embodiment of the invention, the phase transition layer may be such that a phase transition occurs to a substance that has an increased electrical conductivity by reacting with the hydrogen ion.

In the biosensor according to one exemplary embodiment of the invention, the phase transition layer may contain one or more selected among VO₂, WO3, hydrogenated amorphous silicon, MoS₂, and WS₂.

In the biosensor according to one exemplary embodiment of the invention, the phase transition layer may contain VO₂ and may be such that VO₂ reacts with hydrogen ions transferred through the hydrogen ion transfer layer to undergo a phase transition to metallic HₓVO₂ (0<x≤1).

In the biosensor according to one exemplary embodiment of the invention, at least one of the first electrode and the second electrode may be made of a substance having a lower work function than a lower work function of the phase transition layer.

In the biosensor according to one exemplary embodiment of the invention, the first electrode and the second electrode may contain one or more selected among In, Al, and Au.

In the biosensor according to one exemplary embodiment of the invention, the first electrode may contain In, and the second electrode may contain Au.

In the biosensor according to one exemplary embodiment of the invention, a target substance may include one or more selected from substances recognized as substrates of a redox enzyme to accompany a generation of a hydrogen ion, a neurotransmitter, a subject substance and a metabolic byproduct involved in a metabolic pathway or muscle metabolism, and a substance associated with a citric acid cycle (Krebs cycle) that maintain a cellular energy balance.

In the biosensor according to one exemplary embodiment of the invention, a device that applies a voltage between the first electrode and the second electrode, and an ammeter that detects a current change between the first electrode and the second electrode may be further included.

Another aspect according to the invention for achieving the above-described object may be a method of detecting a target substance using the biosensor according to the invention, where the method includes:
bringing a specimen containing a target substance into contact with the reaction layer;
generating a hydrogen ion by a physical and/or chemical interaction between the target substance and the reaction layer;
transferring a generated hydrogen ion to a phase transition layer through the hydrogen ion transfer layer;
reacting the transferred hydrogen ion in the phase transition layer to cause a phase transition of the phase transition from an insulating state to a metallic state; and
detecting a change in a current that flows through the phase transition layer.

### BRIEF DESCRIPTION OF THE DRAWINGS

FIG. 1 shows a structure and a circuit diagram of a biosensor according to a first embodiment of the invention;

FIG. 2 illustrates how electron supply from indium (In), a metal with a low work function, accelerates hydrogen ion diffusion to VO₂ in the biosensor.

FIG. 3 is a conceptual view that describes the hydrogen-current amplification principle in heterojunction laminated structure of a GDH-SiO₂-VO₂, which constitutes the biosensor according to the Example of the invention;

FIG. 4 shows the band gap collapse in HₓVO₂ through hydrogen-induced electron doping, leading to an insulating-to-metallic transition;

FIG. 5 shows the results obtained by measuring the amplification state of the current over time in a case where a liquid droplet (1 μl) of a 0.1 M glutamate solution is introduced under the conditions of presence of a glutamate dehydrogenase layer (w/GDH) and absence of a glutamate dehydrogenase layer (w/o GDH);

FIG. 6 shows the results obtained by measuring the amplification state of the current depending on a concentration of a glutamate solution contained in a specimen, under the conditions of presence of a glutamate dehydrogenase layer (w/GDH) and absence of a glutamate dehydrogenase layer (w/o GDH);

FIG. 7 shows current-voltage curves that are obtained in a case of applying a specimen containing glutamate and specimens containing other substances;

FIG. 8 shows the results obtained by comparing the performance of biosensors (red star) according to Examples, and the limits of detection, response times, and amplification rates of an enzyme biosensor (black circle), a glucose biosensor (green circle), and a non-adrenal biosensor (blue circle);

FIG. 9A is a schematic view of an optogenetic technique in which irradiation is carried out in the hippocampus area with blue light illumination that activates neuronal cells expressing channel rhodopsin (ChR2) so that glutamate is released at the synapse. FIG. 9B shows an experimental device for monitoring, during irradiation with a blue light laser, the dynamics of glutamate responding to the light in live neurons (the dissected ChR2 expressing hippocampal tissue from a mouse brain is mounted in a sensing region of the biosensor between the source electrode and the drain electrode, and then the release of glutamate is monitored at the local synaptic site (see lower part)) by using a biosensor according to the Example of the invention;

FIG. 10 shows the results obtained by measuring a change in current over time, depending on the time at which the dynamics of glutamate responding to light is monitored;

FIG. 11A shows a superimposed profile of changes in current according to frequency, where for comparison, the glutamate-induced current signal is obtained by subtracting the background signal caused by the accumulation of natural glutamate release, and FIG. 11B shows the results obtained by continuously monitoring the changes in glutamate-induced current during light irradiation at various duration times (1 second, 10 seconds, and 100 seconds) (the inserted graph shows a superimposed profile of changes in current depending on the irradiation time);

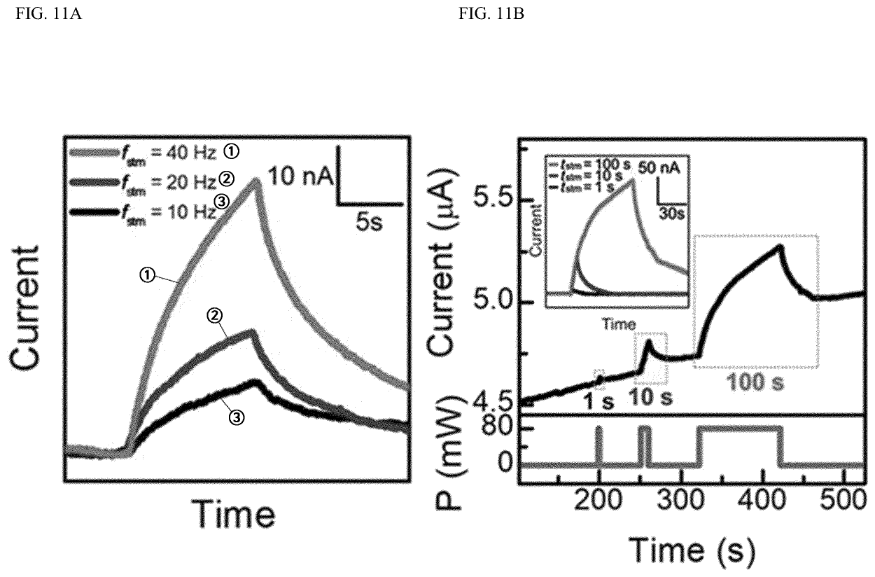

FIG. 12A shows a current-voltage curve in a case where a biosensor in which a specimen containing glutamate is applied to a biosensor to which a WO₃ layer is applied as a phase transition layer, and FIG. 12B shows the results obtained by measuring a change in resistance over time under the conditions of presence of a glutamate dehydrogenase layer (w/glutamate) and absence of a glutamate dehydrogenase layer (w/o glutamate);

FIG. 13 shows the results obtained by measuring the amplification state of the current in Example 1 using an indium electrode and Example 3 using an aluminum electrode, in a case where a liquid droplet (1 μl) of a 0.1 M glutamate solution is introduced;

FIG. 14 shows the results obtained by measuring the amplification state of the current in a case where a liquid droplet (1 μl) of an acetone solution having a concentration of 10⁻¹ M to 10⁻¹⁷ M is introduced in a case where an alcohol dehydrogenase substance layer is present; and

FIG. 15 shows the results obtained by measuring the amplification state of the current in a case where a liquid droplet (1 μl) of an aldehyde solution having a concentration of 10⁻¹ M to 10⁻¹⁷ M is introduced in a case where an aldehyde dehydrogenase substance layer is present.

### DETAILED DESCRIPTION OF THE EXEMPLARY EMBODIMENTS

Hereinafter, with reference to the attached drawings, examples of the invention will be described in detail so that a person skilled in the art can easily carry out the examples of the invention. However, the invention may be embodied in various forms different from each other and thus is not limited to the examples described herein.

In addition, in order to clearly describe the invention in the drawings, portions unrelated to the description have been omitted, and similar reference numerals for drawings have been attached I to similar portions throughout the specification.

Throughout the present specification, in a case where a certain portion is said to be “connected” to another portion, it includes not only a case of “directly connected” but also a case of “electrically connected” with another element being interposed therebetween.

Throughout the present specification, in a case where a certain member is described to be located “on”, “at an upper part”, “at an upper end”, “under”, “at a lower part”, or “at a lower end” of another member, this includes not only a case where a member is in contact with another member but also a case where another member exists between the two members.

Throughout the present specification, in a case where a certain part is said to “include” a certain constitutional element, it means that other constitutional elements may be further included rather than excluding other constitutional elements, unless the context specifically states otherwise.

Terms such as “about” and “substantially” which are used in the present specification are used to mean to be at the numerical value or close to the numerical value in a case where allowable errors for intrinsic manufacturing and intrinsic substances are provided for the mentioned meaning, and they are used to prevent an unscrupulous infringer from unfairly utilizing the disclosed content in which accurate or absolute numerical values are mentioned to aid the understanding of the invention. In addition, throughout the present specification, the term “step of ˜ing” or “step of ˜” does not mean “step for ˜.”

Throughout the present specification, the term “a combination thereof” included in the Markush form means a mixture or combination of one or more selected from the group consisting of constituent elements, which is described in the Markush form expression, and it is used to mean that one or more selected from the group consisting of the constituent elements are included. Throughout the present specification, the description “A and/or B” means “A or B, or A and B”.

### First Embodiment

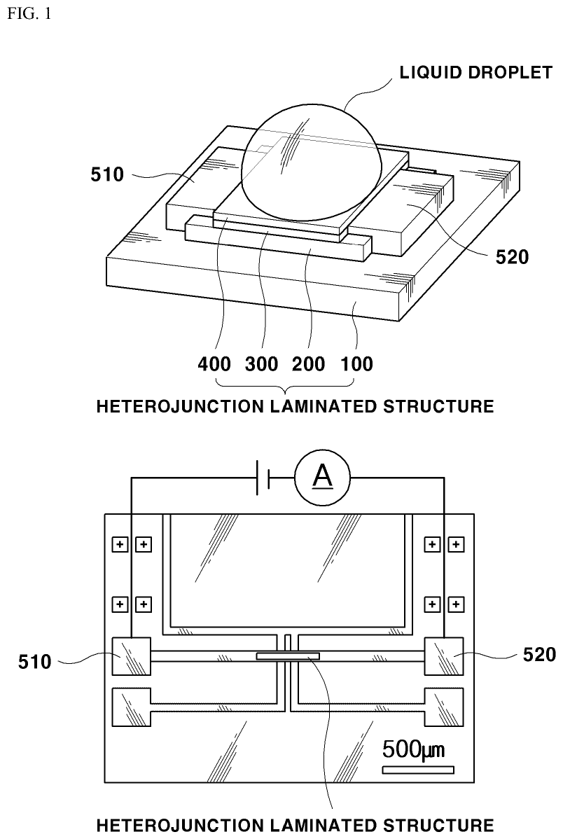

Referring to FIG. 1, a first embodiment of the invention is a biosensor formed on a substrate 100, which is configured to include a phase transition layer 200 formed on the substrate 100, a hydrogen ion transfer layer 300 formed on the phase transition layer 200, a reaction layer 400 formed on the hydrogen ion transfer layer 300, a first electrode 510 formed on one side of the reaction layer 400, and a second electrode 520 formed on the other side of the reaction layer 400.

The substrate 100 may contain one or more selected among glass, silicon, a metal, a polymer material, and a ceramic, and any substrate may be used as long as it has such insulating properties that do not interfere with the detection of the current flowing through the phase transition layer 200, or it has an insulating layer.

In the phase transition layer 200, a phase transition to a substance having an increased electrical conductivity may be caused due to a hydrogen ion transferred through the hydrogen ion transfer layer 300. In a case where the reaction layer 400 generates a hydrogen ion through a reaction with a target substance, the generated hydrogen ion is transferred to the phase transition layer 200 through the hydrogen ion transfer layer 300, which increases the electrical conductivity of the phase transition layer 200 due to the phase transition in the phase transition layer 200. As a result, the current flowing through the phase transition layer 200 increases, which allows the phase transition layer 200 to amplify the signal of the target substance by more than 100 times for detection, thereby allowing the signal to be detected.

The phase transition layer 200 contains a substance that undergoes a phase transition to a substance that has increased electrical conductivity by reacting with the hydrogen ion, and such a substance may contain, for example, one or more selected among VO₂, WO₃, hydrogenated amorphous silicon, MOS₂, and WS₂, and may preferably contain vanadium dioxide (VO₂).

Vanadium dioxide (VO₂) reacts with a hydrogen ion to undergo a phase transition from insulating VO₂ to metallic HₓVO₂ (0<x≤1), thereby significantly reducing the electrical resistance of the phase transition layer 200 (greatly increasing electrical conductivity), and in this process, it is possible to greatly increase sensitivity to the target substance.

If the thickness of the phase transition layer 200 is less than 5 nm, a uniform thin film may not be formed, and if the thickness of the phase transition layer 200 exceeds 20 nm, it is difficult to grow as an epitaxial thin film, and thus it is preferable to form the phase transition layer 200 in a range of 5 to 20 nm.

The hydrogen ion transfer layer 300 is a layer that transfers the hydrogen ion generated in the reaction layer 400 to the phase transition layer 200. The hydrogen ion transfer layer 300 may be used without limitation as long as it is a substance having a structure that is capable of transferring a hydrogen ion. However, to increase the reliability and durability of the biosensor, it is desirable to transfer only a hydrogen ion while preventing a liquid from being permeated and transferred from the reaction layer 400 in contact with a liquid specimen to the phase transition layer 200, and thus it is desirable to have a porous structure that has liquid impermeability and is capable of transferring a hydrogen ion.

In addition, the hydrogen ion transfer layer 300 is preferably made of a substance having proton conductivity, which enables ion transfer while maintaining electronic insulation in order to prevent unintended electron conduction.

Such a substance may include, for example, a proton conductive solid oxide such as a silicon oxide, a proton conductive polymer substance such as Nafion, perfluorosulfonic acid (PFSA), or a sulfonated polyether ether ketone (SPEEK), a perovskite proton conductor, a hydrogen-bonded organic framework (HOF) that facilitates proton hopping due to the hydrogen bonding thereof and has a low electronic conductivity, thereby being capable of acting as an insulator, a substance functionalized with an acidic group or a protonic molecule such as SO₃H, PO₃H₂, imidazole, or histamine, a proton conductive ceramic, and the like.

The hydrogen ion transfer layer 300 may preferably contain amorphous SiO₂.

If the thickness of the hydrogen ion transfer layer 300 is less than 5 nm, the hydrogen ion transfer layer 300 may grow to have a shape of a ununiform thin film, and if the hydrogen ion transfer layer 300 exceeds 10 nm, the high-speed movement of the hydrogen ion may be hindered, and thus it is preferable that the hydrogen ion transfer layer 300 is formed to have a thickness in a range of 5 to 10 nm.

The reaction layer 400 is configured to include a substance generating a hydrogen ion through a physical and/or chemical interaction with a target substance in a specimen. For example, the reaction layer 400 may contain a redox enzyme that accompanies the generation of the hydrogen ion. The redox enzyme may include a dehydrogenase, a peroxidase, a reductase, an oxidase, an oxygenase, a hydroxylase, and the like.

In the present specification, the “target substance” refers to a substance to be detected, which is present in a specimen, and it is a substance generating a hydrogen ion through a physical and/or chemical interaction with a substance that constitutes the reaction layer 400.

The target substance may be a substance that is recognized as a substrate of a redox enzyme to accompany a generation of a hydrogen ion, a neurotransmitter including glutamate, a subject substance and a metabolic byproduct involved in a metabolic pathway or muscle metabolism, a substance associated with a citric acid cycle (Krebs cycle) that maintain a cellular energy balance, and the like.

If the thickness of the reaction layer 400 is less than 5 nm indicates that enzyme coating has not been achieved, and the enzyme aggregates and cannot function if the thickness of the reaction layer 400 exceeds 40 nm. Therefore, it is preferable for the reaction layer 400 to have a thickness of 5 to 40 nm and is more preferable to have a thickness of 10 to 20 nm.

The first electrode 510 and the second electrode 520 are formed at both end parts of the phase transition layer 200 in order to measure a change in current in the phase transition layer 200. However, without forming the first electrode 510 and the second electrode 520, a power supply, an ammeter, and the like may be directly connected to both end parts of the phase transition layer 200.

It is preferable that at least one among the first electrode 510 and the second electrode 520 includes a substance having a lower work function than a lower work function of the phase transition layer 200. This is because if the work function of the electrode is lower than the work function of the substance that constitutes the phase transition layer 200, the detection may be rapidly carried out since electrons, which may accelerate the penetration of the hydrogen ion transferred to the phase transition layer 200 through free diffusion in the hydrogen ion transfer layer 300, are supplied.

For example, if VO₂ is applied to the phase transition layer 200, the first electrode 510 may contain Al and/or In, which has a lower work function than VO₂, and the second electrode 520 may include a substance containing Au. In this case, the second electrode 520 may also include the same substance as the substance of the first electrode 510.

As illustrated in the lower part of FIG. 1, a device for applying a voltage and an ammeter for detecting a change in current between the first electrode 510 and the second electrode 520 may be included between the first electrode 510 and the second electrode 520. By applying a voltage in this way, a change in current passing through the phase transition layer 200 is detected, whereby the target substance contained in the specimen is detected.

### Second Embodiment

A second embodiment of the invention is a method of detecting a target substance using the biosensor according to the first embodiment, and the method includes: bringing a specimen containing a target substance into contact with the reaction layer 400; generating a hydrogen ion by a physical and/or chemical interaction between the target substance and the reaction layer 400; transferring a generated hydrogen ion to a phase transition layer 200 through the hydrogen ion transfer layer 300; reacting the transferred hydrogen ion in the phase transition layer 200 to cause a phase transition from an insulating substance to a metallic substance; and detecting a change in a current that flows through the phase transition layer.

In the second embodiment of the invention, a hydrogen ion is s generated through an interaction with a target substance, and a phase transition from an insulating substance to a metallic substance is induced through a reaction with the generated hydrogen ion. In addition, through a current change due to such a phase transition, the conversion from the hydrogen ion to the current is achieved, which makes it possible to carry out the detection of the target substance together with the amplification of the detection signal, thereby obtaining a detection sensitivity that is difficult to be achieved in a biosensor.

In the present specification, the term “detection” may refer to the discovery or confirmation of the presence of a target substance and may include, for example, identifying a target substance or quantifying a target substance in a specimen. The above-described detection method may be a method of measuring a change in current or voltage, which occurs due to a phase transition to a metallic state by a reaction between the generated hydrogen ion through a physical and/or chemical interaction of a target substance in reaction layer 400 and the substance in phase transition layer 200.

### Example 1

A biosensor according to the Example of the invention was a biosensor for detecting glutamate which is a neurotransmitter, as a target substance, and it was formed to have the following heterojunction laminated structure on a sapphire (Al₂O₃) substrate 100.

First, a crystallized VO₂ layer was formed on the sapphire substrate as the phase transition layer 200. The VO₂ layer was allowed to grow from a V₂O₅ target using pulsed laser deposition (PLD) in an oxygen atmosphere, and the thickness of the formed VO₂ layer is 20 nm.

A porous amorphous SiO₂ layer was formed on the VO₂ layer as the hydrogen ion transfer layer 300. The amorphous SiO₂ layer was similarly allowed to grow from a SiO₂ target by using PLD under an oxygen atmosphere, and the thickness of the formed amorphous SiO₂ layer is 5 nm. On the other hand, the amorphous SiO₂ layer has nano porosity and has liquid impermeability, and thus it protects the VO₂ layer located at the lower part, from the external aqueous environment.

On a surface of the amorphous SiO₂ layer, the reaction layer 400, which is a substance layer containing glutamate dehydrogenase (GDH), may recognize a target and selectively produce a hydrogen ion. Under the conditions of the chemical solution process of three steps (a modification of a SiO₂ surface through APTS, a treatment with glutaraldehyde, and an enzyme immobilization reaction through the Schiff base reaction), a covalent bond of the enzyme was formed on the SiO₂ surface, and the thickness of the formed glutamate dehydrogenase layer is 10 to 20 nm.

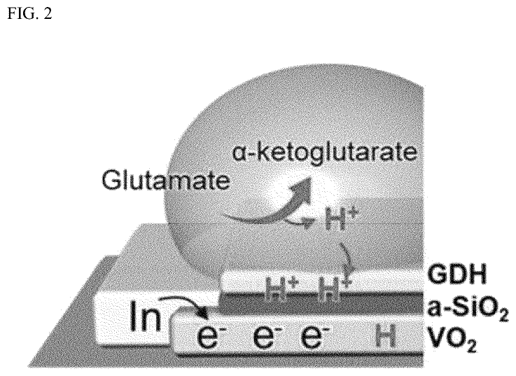

At one end part of the phase transition layer 200, an indium (In) electrode was formed by a thermal evaporation method to have a thickness of 50 nm under a high vacuum, and at the other end part of the phase transition layer 200, a gold (Au) electrode was also formed by a thermal evaporation method to have a thickness of 50 nm under a high vacuum. For phase transition of insulating VO₂ to metallic HₓVO₂, an electron should be bonded to a hydrogen ion. The spontaneous oxidation due to a lower work function of indium (about 4.09 eV or less) than VO₂ may provide an electron to VO₂ (see FIG. 2), which may accelerate the penetration of the hydrogen ion into VO₂, thereby enabling faster responsiveness.

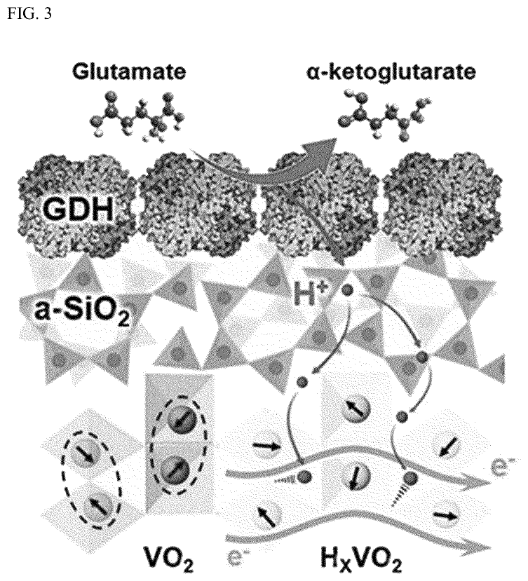

FIG. 3 is a conceptual view describing the hydrogen-current amplification principle in a heterojunction laminated structure of glutamate dehydrogenase (GDH)—amorphous SiO₂-crystalline VO₂, which constitutes the biosensor according to the Example of the invention. Referring to 3, due to glutamate recognition based on the three-dimensional stereospecificity, a GDH layer which is half-layered does not react with other molecules even in a complex mixture, and a dehydrogenase reaction selective for glutamate occurs. Due to the dehydrogenase reaction, glutamate is converted into a hydrogen ion and α-ketoglutarate. The generated hydrogen ion is easily absorbed to the amorphous SiO₂ surface located below the GDH layer then to be quickly transferred to the VO₂ layer located at a lowermost part through an ultrathin porous layer. When the hydrogen ion is transferred to the VO₂ layer, the hydrogen ion is inserted along an empty tunnel in the pseudo Rutile crystal lattice, which results in the phase transition from an insulating substance (VO₂) to a metallic substance (HₓVO₂).

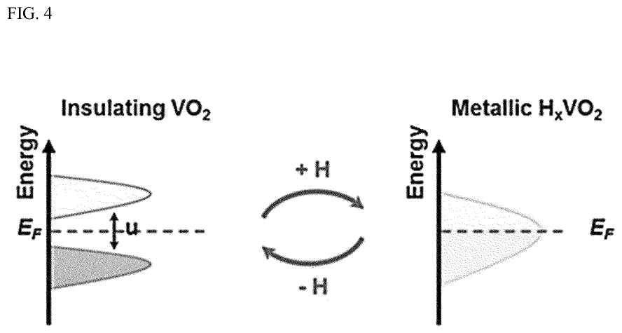

As the insertion of the hydrogen ion aligns with the ion-electron bonding, electrons may flow through the VO₂ lattice, and electron doping drastically collapses the correlation-based band gap together with electron occupation in the conduction band (FIG. 4). Through such a transition from an insulator to a metal, electrical resistivity may be reduced by a factor of up to 104 times, and the current signal may be greatly amplified during glutamate detection.

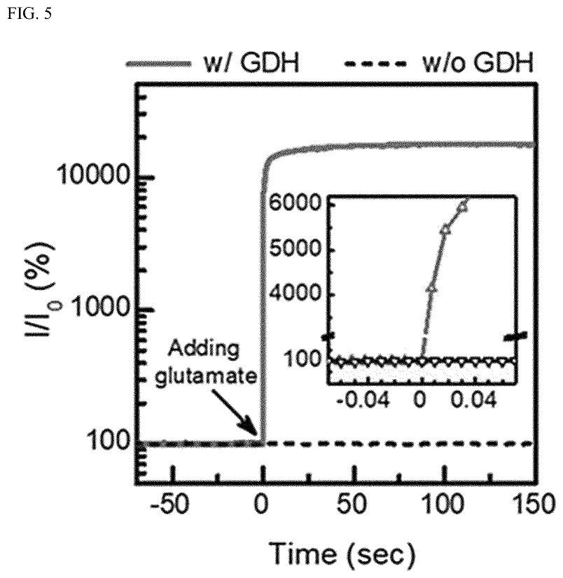

FIG. 5 shows the results obtained by measuring the amplification state of the current over time in a case where a liquid droplet (1 μl) of a 0.1 M glutamate solution is introduced under conditions of presence of a glutamate dehydrogenase layer in the reaction layer (w/GDH) and absence a glutamate dehydrogenase layer (w/o GDH). As shown in FIG. 5, for measuring the current at millisecond intervals, the current amplification was quite large, and a rapid increase in current was observed in real-time immediately after the introduction of the liquid droplet. The amplified current (I) within 50 ms increased by 10,000 times or greater with respect to the reference (I₀) and finally, finally reached 17,200% of I/I₀ (red solid line). Meanwhile, when the GDH layer was not formed, there was a negligible current change since the enzyme reaction did not occur, and a current increase of 0.016% was observed after 150 seconds (shown as a black dotted line in the drawing).

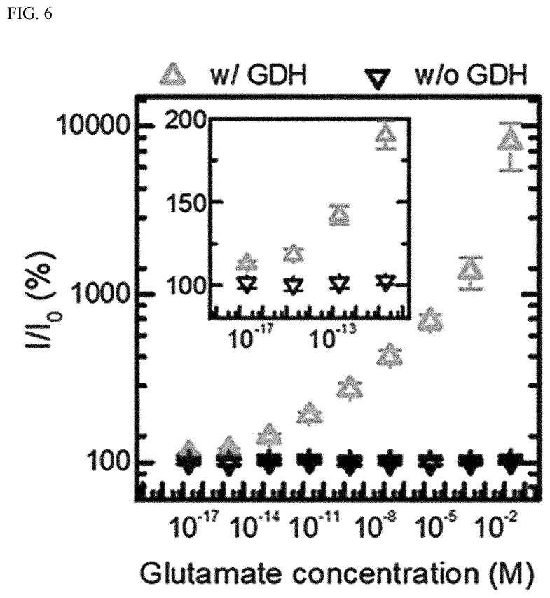

Next, the glutamate solution was continuously diluted from 10⁻¹ M to 10⁻¹⁷ M to carry out a real-time current measurement. FIG. 6 shows the results obtained by measuring the amplification state of the current depending on a concentration of a glutamate solution contained in a specimen, under the conditions of presence of a glutamate dehydrogenase layer (w/GDH) and absence of a glutamate dehydrogenase layer (w/o GDH). As confirmed in FIG. 6, if GDH was absent, there was no change in current when glutamate was introduced (black data). However, if GDH was present, current amplification was observed in a wide range of the tested glutamate concentrations (red data). In a case of monitoring a current change for a period of 300 seconds or more, an increase of 5% in I/I₀ was induced even at a very dilute glutamate concentration of 10⁻¹⁷ M, and thus an electrical signal capable of detecting even a level of dozens of glutamate molecules may be generated. That is, it can be seen that the biosensor according to the Example makes it possible to obtain ultra-high sensitivity characteristics through hydrogen penetration and the amplification of the electric signal driven by the penetrated hydrogen.

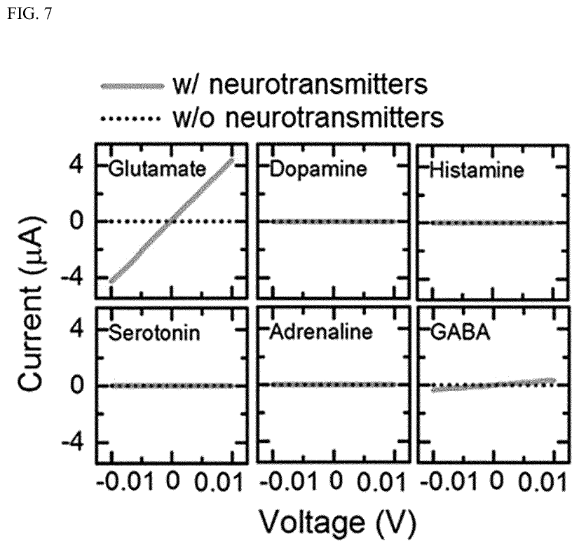

Next, in order to verify the selectivity of the biosensor according to the Example for a target substance, the comparison was carried out between substances including dopamine, histamine, serotonin, adrenaline, and gamma-aminobutyric acid (GABA). FIG. 7 shows current-voltage curves that are obtained when a specimen containing glutamate and specimens containing other substances are respectively applied. Although the tested substances were similar to glutamate in terms of size and chemical structure, as confirmed in FIG. 7, no significant differences were observed regarding the current for the 0.1 M solution. However, GABA was less affected by positional specificity of GDH, and thus GABA was observed to increase I/I₀ two times. However, this is much smaller as compared with the 170-fold increase in I/I₀ induced by glutamate, which is the target substance. Therefore, the increase was still practically small. That is, it can be seen that the biosensor according to the Example shows extremely excellent selectivity for a target neurotransmitter.

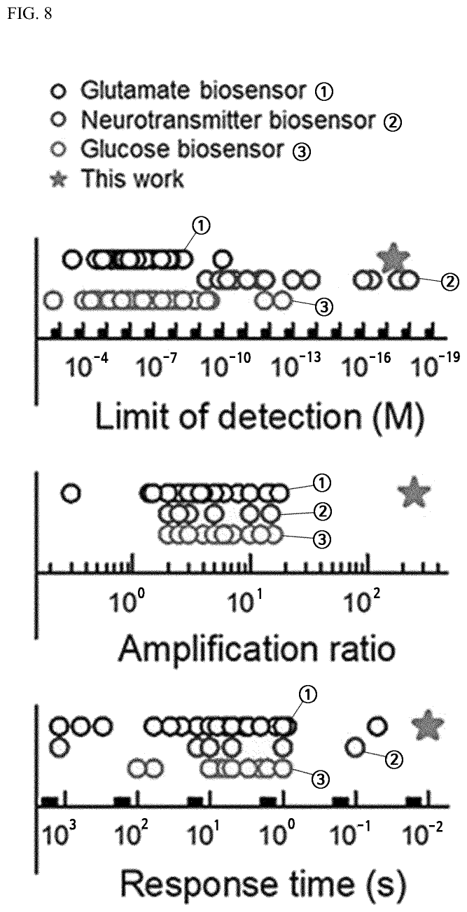

FIG. 8 shows the results obtained by comparing the characteristics of the biosensor according to the Example, with those of previously known electrochemical sensors and FET sensors (a glutamate biosensor, a neurotransmitter biosensor, and a glucose biosensor). As confirmed in FIG. 8, the biosensor according to the Example marked with an asterisk (indicated as “this work” in the drawing) showed characteristics such that the biosensor according to Example has an lowest limit of detection, has a highest signal amplification rate, and has a fastest response speed, demonstrating overall superior performance as compared with the biosensors in the related art.

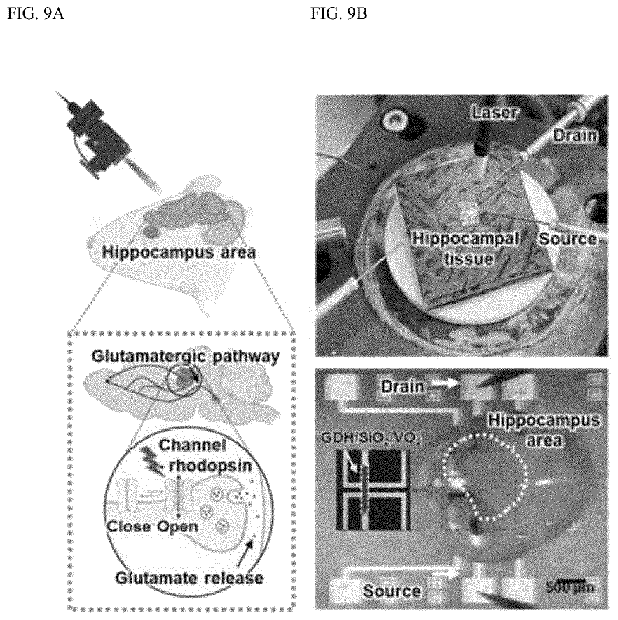

Next, using the biosensor according to Example, the dynamics of glutamate responding to light in real-time was monitored in a brain tissue of a small mouse (see FIG. 9A and FIG. 9B).

For optogenetic neural stimulation, channel rhodopsin (ChR2), which is a blue light-gated ion channel, was intentionally expressed in a hippocampus area composed of highly dense bundles of nerve cells. ChR2, which was overexpressed under external blue light illumination (Λ=473 nm; power=80 mW), carried out cell depolarization and propagated action potentials. This induced living neuron cells to release glutamate and continuously activated other neurons through the glutamatergic synaptic pathway. To monitor synaptic glutamate release, the cerebral cortex region of the mouse brain was arranged in the sensing region having a heterojunction laminated structure of glutamate dehydrogenase (GDH)-amorphous SiO₂-crystalline VO₂ between the source electrode (first electrode) and the drain electrode (second electrode).

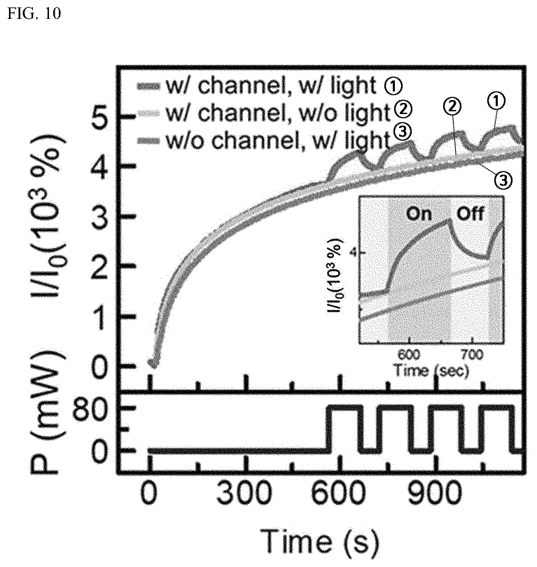

As a result, as shown in FIG. 10, the release of glutamate at the neural synapse was detected in real-time due to the hydrogen-current amplification induced by the glutamate synchronized with blue light illumination. In a case where a brain slice was in an artificial cerebrospinal fluid (aCSF), spontaneous glutamate release occurred at a sub-micromolar level in the extracellular environment and was easily recognized as an immediate increase in current. Even after the initial explosion, the gradual increase in current was measured as the accumulation of natural glutamate leakage based on the cell death over time. However, in a case where neurons with overexpressed ChR2 were stimulated by blue laser light for 600 seconds after sample loading, the current profile over time clearly included an additional logarithmic curve and recorded the release of glutamate which responded to light at the neuron synapse (red line). In a case where the light stimulation disappeared, the current signal decreases exponentially due to the reabsorption of neural glutamate, and the cyclical 100-second light irradiation (blue line) showed a repeated pattern of current increase and decrease. Through this, the stable and reproducible sensing of dynamics of glutamate generation and decay by the biosensor according to Example was verified.

Next, the optical pulse frequency and the period of the optical pulse stimulation were adjusted to measure the rate of glutamate release which was optogenetically induced. In this case, in order to investigate the increase in current due to light pulses during glutamate monitoring, the frequency of blue light was initially changed to have the same intensity at a constant duty cycle.

As shown in 11a, the glutamate-induced current signal showed such a tendency that the current increased in proportion to the optical frequency at 10 Hz, 20 Hz, and 40 Hz. Similarly, as shown in 11b, the results were such that the current signal induced by glutamate increased depending on the period of the light pulse stimulation.

### Example 2

Example 2 is such that only a substance applied to the phase transition layer 200 constituting the biosensor according to Example 1 is different, and the others are the same.

In Example 2, a WO₃ layer was applied as the phase transition layer 200. The WO₃ layer was allowed to grow from a WO₃ target using pulsed laser deposition (PLD) in an oxygen atmosphere, and the thickness of the formed WO₃ layer is 30 nm.

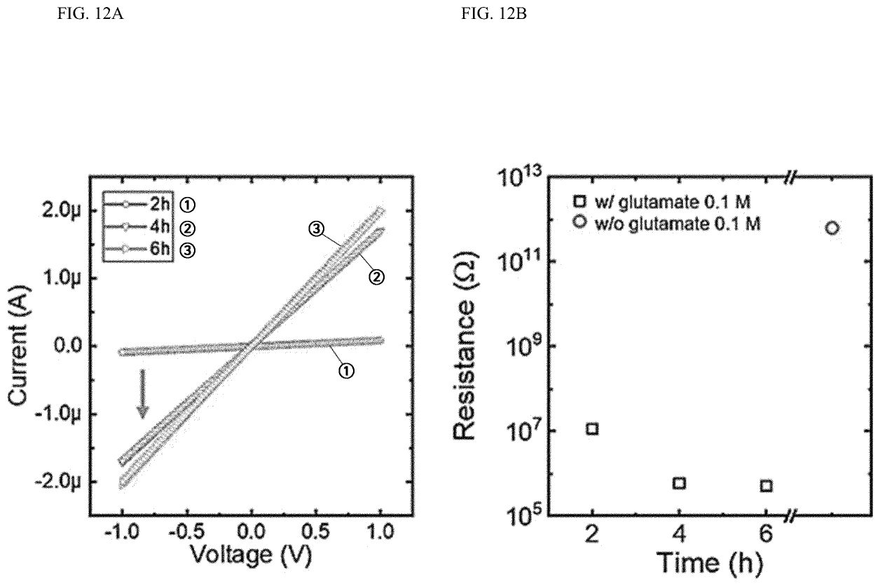

FIG. 12A shows a current-voltage curve in a case where a biosensor in which a specimen containing glutamate is applied to a biosensor to which a WO₃ layer is applied as a phase transition layer. FIG. 12B shows the results obtained by measuring a change in resistance over time under the conditions of presence of a glutamate dehydrogenase layer (w/glutamate) and absence of a glutamate dehydrogenase layer (w/o glutamate).

As confirmed in FIG. 12A and FIG. 12B, when WO₃ was used as the phase transition layer (200) not only showed a result of reduction by approximately 100 times as compared with the initial resistance but also was accompanied by an increase in absorbance due to the decrease in resistance, which further facilitates the real-time measurement.

### Example 3

Example 3 checks whether the indium (In) electrode formed at one end part of the phase transition layer 200 constituting the biosensor according to Example 1 may be formed as an aluminum (Al) electrode.

The aluminum (Al) electrode was formed at one end part of the phase transition layer 200 by a thermal evaporation method to have a thickness of 50 nm under a high vacuum.

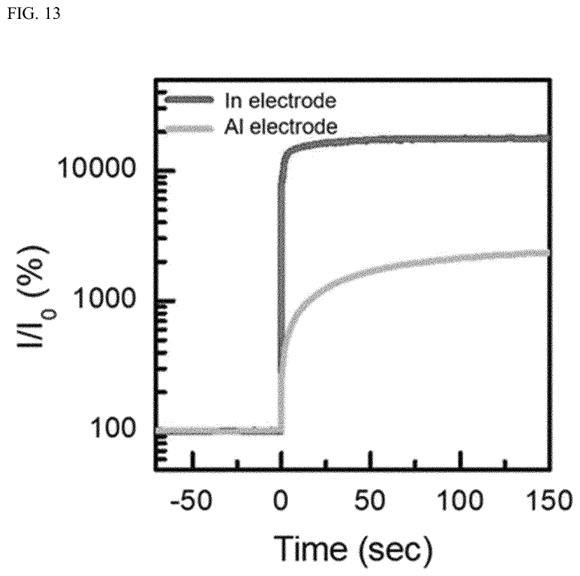

FIG. 13 shows the results obtained by measuring the amplification state of the current in Example 1 using an indium electrode and Example 3 using an aluminum electrode, when a liquid droplet (1 μl) of a 0.1 M glutamate solution is introduced.

As confirmed in FIG. 13, it can be seen that aluminum (Al) may be also applied to a biosensor although the range of current change decreases as compared with indium (In) in a case where aluminum (Al) was applied instead of indium (In) as an electrode substance for electron supply.

### Example 4

Example 4 is such that a substance layer containing alcohol dehydrogenase (ADH) instead of the glutamate dehydrogenase (GDH) is formed as the reaction layer 400 constituting the biosensor of Example 1, and the remaining configuration is the same as in Example 1.

For the substance layer of the enzyme, a covalent bond of the enzyme was formed on the SiO₂ surface by the same chemical solution process as in Example 1 (a modification of a SiO₂ surface through APTS, a treatment with glutaraldehyde, and an enzyme immobilization reaction through the Schiff base reaction).

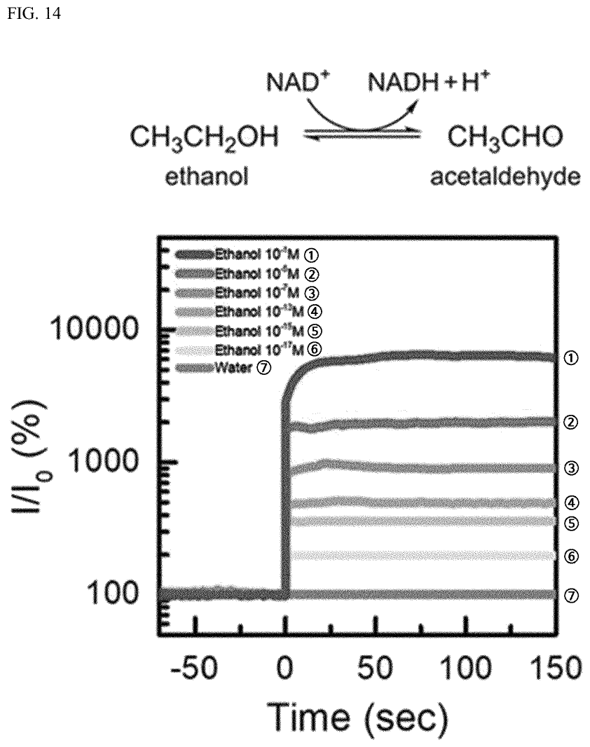

FIG. 14 shows the results obtained by measuring the amplification state of the current in a case where a liquid droplet (1 μl) of an acetone solution having a concentration of 10⁻¹ M to 10⁻¹⁷ M is introduced in a case where an alcohol dehydrogenase substance layer is present.

As confirmed in FIG. 14, a biosensor in which an alcohol dehydrogenase substance layer is formed can be used to detect a target substance, ethanol.

### Example 5

Example 5 is such that a substance layer containing aldehyde dehydrogenase instead of the glutamate dehydrogenase (GDH) is formed as the reaction layer 400 constituting the biosensor of Example 1, and the remaining configuration is the same as in Example 1.

The substance layer of the enzyme was formed by the same chemical solution process method as in Example 1.

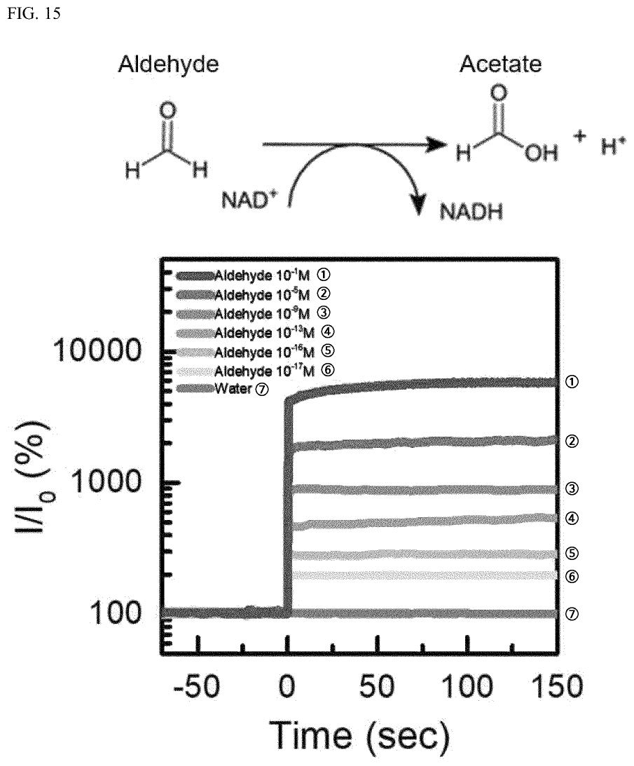

FIG. 15 shows the results obtained by measuring the amplification state of the current in a case where a liquid droplet (1 μl) of an aldehyde solution having a concentration of 10⁻¹ M to 10⁻¹⁷ M is introduced in a case where an aldehyde dehydrogenase substance layer is present.

As confirmed in FIG. 15, a biosensor in which an aldehyde dehydrogenase substance layer is formed can be used to detect a target substance, aldehyde.

What is claimed is:

1. A biosensor comprising:
a reaction layer that generates a hydrogen ion through a physical and/or chemical interaction with a target substance in a specimen;
a hydrogen ion transfer layer that is formed on one side of the reaction layer to transfer the hydrogen ion generated in the reaction layer; and
a phase transition layer that is formed on one side of the hydrogen ion transfer layer, in which a phase of a substance changes due to the hydrogen ion transferred from the hydrogen ion transfer layer.

2. The biosensor according to claim 1, further comprising:
a first electrode that is formed on one side of the phase transition layer; and
a second electrode that is formed on the other side of the phase transition layer.

3. The biosensor according to claim 1,
wherein said reaction layer contains a redox enzyme.

4. The biosensor according to claim 3,
wherein said reaction layer contains one or more selected among a dehydrogenase, a peroxidase, a reductase, an oxidase, an oxygenase, and a hydroxylase.

5. The biosensor according to claim 1,
wherein said hydrogen ion transfer layer contains a substance having proton conductivity that enables ion transfer while maintaining electronic insulation.

6. The biosensor according to claim 5,
wherein said hydrogen ion transfer layer contains one or more selected among a proton conductive solid oxide, a proton conductive polymer substance, a perovskite proton conductor, a hydrogen-bonded organic framework (HOF), a substance functionalized with an acidic group or a protonic molecule, and a proton conductive ceramic.

7. The biosensor according to claim 1,
wherein said hydrogen ion transfer layer has liquid impermeability.

8. The biosensor according to claim 5,
wherein said silicon oxide is an amorphous SiO₂.

9. The biosensor according to claim 1,
Wherein, in said phase transition layer, a substance reacts with said hydrogen ion to have a phase transition to a substance having an increased electrical conductivity.

10. The biosensor according to claim 1,
wherein said phase transition layer contains one or more selected among VO₂, WO₃, hydrogenated amorphous silicon, MoS₂, and WS₂.

11. The biosensor according to claim 1, wherein said phase transition layer contains VO₂, and the VO₂ reacts with the hydrogen ion transferred through said hydrogen ion transfer layer to undergo a phase transition to metallic HₓVO₂ (0<x≤1).

12. The biosensor according to claim 2,
wherein at least one among said first electrode and said second electrode comprises a substance having a lower work function than a lower work function of said phase transition layer.

13. The biosensor according to claim 11,
wherein said first electrode and said second electrode contain one or more selected among In, Al, and Au.

14. The biosensor according to claim 11,
wherein said first electrode contains In, and
said second electrode contains Au.

15. The biosensor according to claim 1,
wherein said target substance contains one or more selected among a substance that is recognized as a substrate of a redox enzyme to accompany a generation of a hydrogen ion, a neurotransmitter, a subject substance and a metabolic byproduct involved in a metabolic pathway or muscle metabolism, and a substance associated with a citric acid cycle (Krebs cycle) that maintain a cellular energy balance.

16. The biosensor according to claim 2, further comprising:
a device that applies a voltage between said first electrode and said second electrode; and
an ammeter that detects a change in current between said first electrode and said second electrode.

17. A detection method of detecting a target substance using the biosensor according to claim 1, the detection method comprising:
bringing a specimen containing a target substance into contact with said reaction layer;
generating a hydrogen ion by a physical and/or chemical interaction between said target substance and said the reaction layer;
transferring the generated hydrogen ion to a phase transition layer through said hydrogen ion transfer layer;
reacting the transferred hydrogen ion in the phase transition layer to cause a phase transition of the phase transition layer from an insulating state to a metallic state; and
detecting a change in a current that flows through said phase transition layer.
